(19) United States
(12) Patent Application Publication
(10) Pub. No.: US 2017/0079905 A1
(43) Pub. Date: Mar. 23, 2017

(54) NATURAL SKIN-CARE PRODUCT

(71) Applicant: Olga RENZ, Mazyr (BY)

(72) Inventor: Olga RENZ, Mazyr (BY)

(21) Appl. No.: 15/330,550

(22) PCT Filed: Apr. 7, 2014
(86) PCT No.: PCT/IB2014/000550

§ 371 Date: Oct. 11, 2016

(51) Int. Cl. A61K 8/97; A61K 36/00; A61K 36/06; A61Q 19/00; A61K 36/02

(52) U.S. Cl. CPC A61K 8/97; A61Q 19/00; A61K 8/975; A61K 36/02; A61K 36/06; A61K 36/00; A61K 2800/412; A61K 2800/413

(57) ABSTRACT

This invention relates to a skin-penetrating skin-care product, which is made of natural, raw ingredients including, among other things, plants, algae and fungi. Skin-care products are generally composed of highly processed components and they often comprise only few isolated nutrients. This invention intends to solve the problem of lack of quality and quantity of nutrients in the skin-care products.

### TECHNICAL FIELD OF THE INVENTION

The present invention relates to the technical field of natural, fresh skin-penetrating skin-care products made of natural, raw ingredients.

### BACKGROUND ART

Skin is the largest organ in the human body. To perform its function and to regenerate, it requires nutrition and rehydration from both inside and out.

### DISCLOSURE OF INVENTION

Skin-care products are generally composed of highly processed components and they often comprise only few isolated nutrients.

This invention intends to solve the problem of lack of quality and quantity of nutrients in skin-care products by presenting a natural, fresh skin-care product, which is made of raw, wholesome ingredients including, among other things, plants, algae and fungi.

Fresh, wholesome ingredients preserve the natural synergy between diverse macronutrients, micronutrients and phytochemicals.

Macronutrients are various proteins, fats and carbohydrates. Micronutrients comprise diverse macrominerals, organic acids, trace minerals and vitamins. It is estimated that in nature there are about twenty-five thousand phytochemicals. Phytochemicals protect the cells from the toxic free radicals, which cause the oxidative stress.

### MODE FOR CARRYING OUT THE INVENTION

This skin-care product is made of natural, raw ingredients including, among other things, plants, algae and fungi.

In order to create a skin-penetrating product, the particle size of the skin-care product is reduced to the micro- or nanosize using a ball mill suitable for micro- or nanogrinding.

### INDUSTRIAL APPLICABILITY

This natural skin-penetrating skin-care product can be used in cosmetic industry and pharmaceutical industry.

1. Skin-care product, characterized by its natural ingredients.

2. Skin-care product according to the claim 1, characterized by its raw ingredients.

3. Skin-care product according to the claim 1, characterized by its minimally processed ingredients.

4. Skin-care product according to the claim 1, characterized by its ability to penetrate the skin.
